(19) United States
(12) Patent Application Publication
(10) Pub. No.: US 2025/0000756 A1
(43) Pub. Date: Jan. 2, 2025

(54) METHOD FOR PRODUCING SOLUTION AND SOLUTION THEREOF

(71) Applicant: BUREAU CO., LTD., Tokyo (JP)

(72) Inventor: Yasunori KASHIWABARA, Tokyo (JP)

(73) Assignee: BUREAU CO., LTD., Tokyo (JP)

(21) Appl. No.: 18/707,650

(22) PCT Filed: Oct. 27, 2022
(86) PCT No.: PCT/JP2022/040115

§ 371 Date: May 6, 2024

(30) Foreign Application Priority Data
Nov. 9, 2021 (JP) 2021-182838

(51) Int. Cl. A61K 8/04; A61Q 5/02

(52) U.S. Cl. CPC A61K 8/046; A61Q 5/02; A61K 2800/432; A61K 2800/592

(57) ABSTRACT

The present invention provides a solution for making lather that has a clearly recognizable color if used, for example, for shampoo or play. The present invention provides a method for producing a solution for making lather, the method comprising: a step for mixing a dye and water with each other; and a step for adding a frothing agent into a dye-containing aqueous solution that is obtained in the above-described step.

### TECHNICAL FIELD

The present invention relates to a solution such as a shampoo.

### BACKGROUND ART

Shampoos, rinses, and conditioners are used for hair (or head) care (washing).

The solution is put in a container body of, for example, a pump device (e.g., a shampoo container). Pushing of a head of the pump device makes the solution in the container body come out through a nozzle outlet. The solution comes out in the form of a lather. At the time, a user could not recognize a color of the lather with the naked eye.

### CITATION LIST

### Patent Literature

[PATENT LITERATURE 1] JP 2021-91611A

### SUMMARY OF INVENTION

### Technical Problem

Upon washing one's hair, adults do not care about a color of lather of a shampoo coming out from a pump device.

Many children (infants) often hate hair washing. In such an occasion, if a lather of a shampoo solution has a color, even the children (infants) disliking the hair washing would be able to enjoy it.

From the aspect, a trial was made to add a coloring material (dye) to the conventional shampoo solution.

Even though an enough amount of coloring material (dye) was added, a color of lather could not be recognized with the naked eye.

In view of the above, to solve the above problem, the present invention provides a solution for making a lather of which color can be clearly recognized with the naked eye upon, for example, hair washing (or playing).

### Solution to Problem

The inventor conducted research and development to unravel why no color could be visually recognized from the lather made of the shampoo solution containing the enough amount of coloring material (dye).

In the repeated trial and error, a lather having a deep color (which can be clearly recognized with the naked eye) was incidentally produced.

At the time, the inventor did not know (the reason) why the lather made of the shampoo solution had the deep color.

The inventor could not specify which lather made of which shampoo solution (shampoo solution how it was produced) had the deep color.

The shampoo solution should have been produced by following the same process as it has been done, though. The inventor could not unravel why the lather made of the shampoo solution had the deep color.

The inventor recalled his memory one by one about the process how the shampoo solution was produced.

Finally, he reminded that the solution which made the deep colored lather was produced through a step of inputting a surfactant (frothing agent) into a dye-containing solution (solution made by mixing a dye and water).

Before the above step, the inventor input the coloring material (dye) and the surfactant together into water at the same time (at once). This is the typical way in mixing procedure.

The inventor did not know why such a difference occurred when changing the order of adding the materials in the mixing (adding) step.

However, it surely happened that the lather having the deep color could be obtained as a result of changing the order of adding the materials in the mixing (adding) step.

In view of the above, the inventor tried to add a dye into a surfactant-containing solution which was made by mixing a surfactant and water. As a matter of course, the same type and the same amount of surfactant and dye as used in the above case were employed. In this case, however, the lather having the deep color as being obtained in the above case could not be achieved.

Results were the same even when a type of surfactant and/or dye were changed, or even when an amount of surfactant and/or dye were changed.

The inventor studied again by putting such results together.

As a result, the inventor came to have an idea as described below.

Initially, the inventor studied it case by case, i.e., a case where a surfactant, a dye, etc., together were added to water at the same time, a case where a dye was added to a surfactant-containing aqueous solution, and a case where a surfactant was added to a dye-containing aqueous solution, for the purpose of unraveling why color concentrations of lathers made of those solutions were different from each other.

As a result, the inventor came to have the following idea.

When a coloring material is added to water, a hydrogen bonding occurs between the coloring material and the water. Thereafter, even if a surfactant is added thercto, a bonding (reaction) hardly occurs between the coloring material (whose hand for bonding (reacting) is occupied by the hydrogen bond) and the surfactant. In view of the above, it was so estimated that the surfactant could effectively work as a frothing agent. Also, it was so estimated that the coloring material could effectively work as a coloring material (development of a color: coloration).

In the case where a surfactant, a coloring material, etc., together are added to water at the same time, a bonding (physical bond or chemical bond) occurs between the surfactant and the coloring material. Therefore, it was so estimated that this lowered the function of the surfactant as a surfactant (a frothing agent). It was so estimated that this lowered the function of the coloring material as a coloring material (development of a color: coloration).

It was so estimated that the case where a dye was added to a surfactant-containing aqueous solution resulted in also the same result as the case where a surfactant, a dye, etc., together were added to water at the same time.

Based on the above knowledge, the present invention was made.

The present invention is directed to a method for producing a solution for making a lather, the method comprising:
a step of mixing a coloring material and water; and
a step of adding a frothing agent to the coloring material-containing aqueous solution obtained by the above step.

The present invention is directed to the method, further comprising:
a step of adding a pH adjuster to the coloring material-containing aqueous solution.

The present invention is directed to the method, further comprising:
a step of adding a glycerin to the coloring material-containing aqueous solution.

The present invention is directed to the method, wherein the solution is neutral or weakly acidic.

The present invention is directed to the method:
wherein the frothing agent is 5-50 pts·mass to the 100 pts·mass of water; and
wherein the coloring material is 0.0001-5 pts·mass to the 100 pts·mass of water.

The present invention is directed to the method,
wherein, regarding the coloring material contained in the solution, the reaction with the water lowers a reaction with the frothing agent to thereby allow existence of unreacted coloring material in the solution.

The present invention is directed to the method, wherein the frothing agent is a surfactant.

The present invention is directed to the method, wherein the surfactant is at least one selected from the group consisting of an anionic surfactant, a cationic surfactant, an amphoteric surfactant, and a nonionic surfactant.

The present invention is directed to the method, wherein the coloring material is a water-soluble coloring material.

The present invention is directed to the method, wherein the solution has a pH of a range between 7.3 and 5.5.

The present invention is directed to the method, further comprising:
a step of causing the solution to contain a pH adjuster;
wherein the pH adjuster is 0.01-1 pts·mass to the 100 pts·mass of water.

The present invention is directed to the method, further comprising:
a step of causing the solution to contain a viscosity modifier;
wherein the viscosity modifier is 5-50 pts·mass to the 100 pts·mass of water.

The present invention is directed to the method, wherein the solution is a non-aerosol type solution which uses no compressed gas for making a lather.

The present invention is directed to the method:
wherein the solution is put in a container body of a device comprising the container body, an outlet, and a pump; and
wherein the pump action causes the solution and the air in the container body to mix each other to eject a colored lather through the outlet.

The present invention is directed to the method, wherein, when a shading (light and shade) of color is shown by gradation of 10 stages, the shading of color of the lather made by the solution is at a level deeper than the 7^{th} stage from the deeper color of the gradation.

The present invention is directed to a solution for making a lather:
wherein the solution contains at least a frothing agent, a coloring material, and water;
wherein there exist chemically unreacted frothing agent and coloring material between the frothing agent and the coloring material in the solution; and
wherein the solution is neutral or weakly acidic.

The present invention is directed to the solution:
wherein the frothing agent is 5-50 pts·mass to the 100 pts·mass of water; and
wherein the coloring material is 0.0001-5 pts·mass to the 100 pts·mass of water.

The present invention is directed to the solution:
wherein, regarding the coloring material contained in the solution, the reaction with the water lowers a reaction with the frothing agent to thereby allow existence of unreacted coloring material in the solution.

The present invention is directed to the solution, wherein the frothing agent is, preferably, a surfactant.

The present invention is directed to the solution, wherein the surfactant is at least one selected from the group consisting of an anionic surfactant, a cationic surfactant, an amphoteric surfactant, and a nonionic surfactant.

The present invention is directed to the solution, wherein the coloring material is, preferably, a water-soluble coloring material.

The present invention is directed to the solution, wherein the solution, preferably, has a pH of a range between 7.3 and 5.5.

The present invention is directed to the solution, wherein the solution is neutral or weakly acidic.

The present invention is directed to the solution:
wherein the solution, preferably, contains a pH adjuster, and
wherein the pH adjuster is, preferably, 0.01-1 pts·mass to the 100 pts·mass of water.

The present invention is directed to the solution:
wherein the solution, preferably, contains a viscosity modifier; and
wherein the viscosity modifier is, preferably, 5-50 pts·mass to the 100 pts·mass of water.

The present invention is directed to the solution, wherein the solution is, for example, a non-aerosol type solution which uses no compressed gas for making a lather.

The present invention is directed to the solution:
wherein the solution is put in a container body of a device comprising, for example, the container body, an outlet, and a pump; and
wherein the pump action causes the solution and the air in the container body to mix each other to eject a colored lather through the outlet.

The present invention is directed to the solution, wherein, when a shading (light and shade) of color is shown by gradation of 10 stages, preferably, the shading of color of the lather made by the solution is at a level deeper than the 7^{th} stage from the deeper color of the gradation.

### Advantageous Effect of Invention

The solution for making a lather of which color can be clearly, visually recognized could be provided in a simple procedure and at low cost.

Use of the solution for a shampoo will cause children and infants to feel happy.

It also will cause children and infants disliking hair washing to do it without hating.

### BRIEF DESCRIPTION OF DRAWINGS

FIG. 1 is a schematic block diagram of a container with a pump in which the solution according to the present invention is put.

### DESCRIPTION OF EMBODIMENTS

A first invention is a method for producing a solution for making a lather.

A second invention is a solution for making a lather.

The solution is specially a solution for making a lather of which color can be clearly recognized with the naked eye. For example, when a shading of color is shown by gradation of 10 stages, the shading of color of the lather made by the solution is at a level deeper than the 7^{th} stage (numerical value: 3) from the deeper color of the gradation. Preferably, the solution is at a level deeper than the 6^{th} stage (numerical value: 4) from the deeper color of the gradation. More preferably, the solution is at a level deeper than the 5^{th} stage (numerical value: 5) from the deeper color of the gradation. Further preferably, the solution is at a level deeper than the 4^{th} stage (numerical value: 6) from the deeper color of the gradation. Setting upper limit is meaningless. The upper limit may be set to, for example, the 4^{th} stage from the deeper color of the gradation.

Here, the gradation of the shading of color is shown as follows: a case where a content of the coloring material is 0 (only pure water) is 0^{th} stage; and a case where the coloring material-saturated aqueous solution (water is pure water) (i.e., a case where a concentration of the coloring material is the most dense) is set to 9^{th} stage. In this manner, the shading of color is shown here by the gradation of 10 stages. That means, a larger value shows a deeper color.

The solution is, for example, a non-aerosol type solution which uses no compressed gas for making a lather. The solution is, for example, a solution which is put in a container body of a device having the container body, an outlet, and a pump, wherein the pump action causes the solution and the air in the container body to mix each other to eject a colored lather through the outlet.

The solution contains at least a frothing agent, a coloring material, and water. There exist chemically unreacted frothing agent and coloring material between the frothing agent and the dye in the solution. For example, regarding the dye contained in the solution, the reaction with the water lowers a reaction with the frothing agent to thereby allow existence of unreacted coloring material in the solution.

The method comprises:
a step of mixing a coloring material (colorant, coloring agent e.g., dye or pigment) and water; and
a step of adding a frothing agent (foaming agent, e.g. surfactant) to the coloring material (colorant, coloring agent e.g., dye or pigment)-containing aqueous solution obtained by the above step.

Preferably, the frothing agent was 5 pts·mass or larger to the 100 pts·mass of water. More preferably, it was 10 pts·mass or larger. Preferably, it was 50 pts·mass or smaller. More preferably, it was 40 pts·mass or smaller. Further preferably, it was 30 pts·mass or smaller.

Preferably, the dye was 0.0001 pts·mass or lager to the 100 pts·mass of water. More preferably, it was 0.001 pts·mass or larger. Further preferably, it was 0.01 pts·mass or larger. Preferably, it was 5 pts·mass or smaller. More preferably, it was 1 pts·mass or smaller. Further preferably, it was 0.1 pts·mass or smaller.

Preferably, the frothing agent (foaming agent) is a surfactant. For example, it is an anionic surfactant. For example, it is a cationic surfactant. For example, it is an amphoteric surfactant. For example, it is a nonionic surfactant. Any type of surfactant will do. Only one type of surfactant may be employed. Two or more types of surfactants also may be employed.

Any anionic surfactant will do. More specifically, for example, the followings may be employed: an N-acylamino acid, a fatty acid, an alkyl ether carboxylic acid, a polyoxyethylene alkyl ether carboxylic acid, an acyl lactic acid, a N-acylmethyalanine, a N-acylsarcosine, a diacylamino acid, and a carboxylate salt type thereof such as a salt thereof, an alkanesulfonic acid, an α-olefin sulfonate, an α-sulpho fatty acid methyl ester, an acylisethionic acid, an alkylsulfosuccinate, a N-acylmethyl taurine, and a sulfate ester salt type thereof such as a salt thereof, an alkyl sulfate ester, a polyoxyethylene alkyl sulfate ester, an alkyl ether sulfate, a polyoxyethylene alkyl ether sulfate, a fatty acid alkanolamide sulfuric ester, and a sulfuric acid salt type thereof such as a salt thereof, and an alkyl phosphate ester, a polyoxyethylene alkylether phosphate, and a phosphate salt type thereof such as a salt thereof.

Any cationic surfactant will do. More specifically, for example, the followings may be employed: an alkyltrimethylammonium salt (e.g., a stearyltrimethylammonium chloride and a lauryltrimethylammonium chloride), an alkylpyridium salt (e.g., a cetylpyridium chloride), a distearyldimethylammonium chloride dialkyldimethylammonium salt, a poly N,N′-dimethyl-3,5-methylene-piperidinium chloride, an alkyl quaternary ammonium salt, an alkyldimethylbenzyl ammonium salt, an alkylisoquinolinium salt, a dialkylmorpholinium salt, a POE-alkyl amine, an alkylamine salt, polyamine fatty acid derivatives, amyl alcohol fatty acid derivatives, a benzalkonium chloride, a benzethonium chloride, etc.

Any amphoteric surfactant will do. More specifically, for example, the followings may be employed: a glycine type amphoteric surfactant (e.g., an alkylglycine salt, a carboxymethyl glycine salt, and a N-acyl aminoethyl-N-(2-hydroxyethyl) glycine salt), an aminopropionic acid type amphoteric surfactant (e.g., an alkyl aminopropionic acid salt and an alkyl iminodipropionate acid salt), an aminoacetic acid betaine type amphoteric surfactant (e.g., an alkyldimethylaminoacetic acid betaine and a fatty acid amidopropyldimethylamino acetic acid betaine), a sulfobetaine type amphoteric surfactant (e.g., an alkylhydroxysulfobetaine), etc.

Any nonionic surfactant will do. More specifically, for example, the followings may be employed: hydrophilic nonionic surfactants such as a POE-sorbitan fatty acid ester (e.g., a POE-solbitan monooleate, a POE-sorbitan monostearate, a POE-solbitan monooleate, and a POE-sorbitan tetraoleate), a POE-sorbitol fatty acid ester (e.g., a POE-sorbit monolaurate, a POE-sorbit monooleate, a POE-sorbit pentaoleate, and a POE-sorbit monostearate), a POE-glycerin fatty acid ester (e.g., a POE-monooleate such as a POE-glycerin monostearate, a POE-glycerin monoisostearate, and a POE-glycerin triisostearate), a POE-fatty acid ester (e.g., a POE-distearate, a POE-monodioleate, and an ethylene glycol distearate), a POE-alkyl ether (e.g., a POE-lauryl ether, a POE-oleylether, a POE-stearyl ether, a POE-behenyl ether, a POE-2-octyldodecyl ether, and a POE-cholestanol ether), a pluronic-type (e.g., a pluronic), a POE/POP-alkyl ether (e.g., a POE/POP-cetyl ether, a POE/POP-2-decyl tetradecyl ether, a POE/POP-monobutyl ether, a POE/POP-hydrogenated lanolin, and a POE/POP-glycerin ether), a tetraPOE/tetraPOP-ethylenediamine condensate (e.g., a tetronic), POE-hardened castor oil derivatives (e.g., a POE-castor oil, a POE-hardened castor oil, a POE-hardened castor oil monoisostearate, a POE-hardened castor oil triisostearate, a POE-hardened castor oil monopyroglutamic acid monoisostearic acid diester, and a POE-hardened castor oil maleic acid), POE-beeswax/lanolin derivatives (e.g., a POE-sorbit beeswax), an alkanolamide (e.g., a coconut oil fatty acid diethanolamide, a lauric acid monoethanolamide, and a fatty acid isopropanolamide), a POE-propylene glycol fatty acid ester, a POE-alkyl amine, a POE-fatty acid amide, a sucrose fatty acid ester, an alkyl ethoxy dimethylamine oxide, a trioleyl phosphoric acid, etc.

The surfactant may be either one of a petroleum-derived synthetic surfactant or a naturally derived surfactant (i.e., soap).

Preferably, the coloring material (colorant, coloring agent e.g., dye or pigment) is a water-soluble coloring material (colorant, coloring agent e.g., dye or pigment). Examples thereof include Red No. 102 (C₂₀H₁₁N₂Na₃O₁₀S₃, 2-hydroxy azonaphthalene-4′, 6, 8-trisulfonic acid trisodium), Red No. 106 (sodium 9-(2,4-disulfonatophenyl)3,6-bis(diethylamino)xanthylium), Blue No. 1 (Brilliant Blue FCF: (Ethyl) [4-[[2-(sodiooxysulfonyl) phenyl] [4-[[3-(sodiooxysulfonyl) benzyl] (ethyl) amino]phenyl]methylene]-2,5-cyclohexadien-1-ylidene] (3-sulfonatobenzyl) aminium), Yellow No. 4 (1H-Pyrazole-3-carboxylic acid, 4, 5-dihydro-5-oxo-1-(4-sulfophenyl)-4-[(4-sulfophenyl)azo]-, trisodium salt), Yellow No. 203 (2-(1,3-Dioxoindan-2-yl) quinolinedisulfonic acid sodium salt), Purple No. 401 (2-N-[(9,10-Dihydro-4-hydroxy-9,10-dioxoanthryl] amino-5-methylbenzenesulfonic acid sodium salt), etc. As a matter of course, examples are not limited thereto.

The solution is used for skin, etc. Therefore, the solution preferably had a pH of a range, for example, between 7.3 and 5.5. Preferably, the pH was between neutral and weakly acidic.

To make the pH put in a range between neutral and weakly acidic, for example, a pH adjuster is used. Examples thereof include an adipic acid, a citrate, a trisodium citrate, a glucono delta lactone, a gluconic acid, a potassium gluconate, a sodium gluconate, a succinic acid, a monosodium succinate, a disodium succinate, a sodium acetate, a DL-tartaric acid, a L-tartaric acid, a DL-potassium bitartrate, a L-potassium bitartrate, a hydrogen sodium carbonate, a glacial acetic acid, a lactic acid, a sodium lactate, a disodium dihydrogen pyrophosphate, a phosphoric acid, a sodium phosphate, a DL-malic acid, etc. As a matter of course, examples are not limited thereto. A typical pH adjuster is a citrate.

The pH adjuster was, for example, 0.01-1 pts·mass to the 100 pts·mass of water.

The solution, preferably, contains a viscosity modifier for a viscosity.

Examples of the viscosity modifier include a pectic acid, a guar gum, a xanthan gum, a tamarind gum, an Arabian gum, a carrageenan, a propylene glycol, a carboxymethyl cellulose (CMC), and a glycerin. The glycerin can be used as a moisturizing agent, a thickening stabilizer, and the like. The glycerin provides skin with a softening feeling and a moist feeling and makes a thin film over the skin to protect the skin from drying by the outside air. Therefore, the use of glycerin is suitable for the application of the present invention.

The viscosity modifier was, preferably, 5-50 pts·mass to the 100 pts·mass of water.

The solution according to the present invention is produced in a manner as follows.

Initially, the coloring material (colorant, coloring agent e.g., dye or pigment) was mixed with (added to) water (e.g., distilled water or purified water). The ratio was as described above. The mixture was subjected to stirring. This made the coloring material (colorant, coloring agent e.g., dye or pigment) well dissolved.

A coloring material-containing aqueous solution in which the coloring material was dissolved was mixed with (added to) the frothing agent (foaming agent, e.g. surfactant). It was subjected to stirring. This made the frothing agent well dispersed.

The pH adjuster, the glycerin, etc., were also mixed with (added to) the dye-containing aqueous solution, as required, together with (or one by one with) the surfactant.

Hereinafter, specific examples are described. The present invention is not limited only to the following examples. It is to be understood that, unless otherwise characteristics of the present invention is not impaired largely, various changes and modifications should be construed as being included in the scope of the present invention.

### Example 1

Red No. 106 of 0.1 pts·mass was added to 100 pts·mass of purified water. Stirring and mixing were performed.

A citrate of 10 pts·mass was added to 100 pts·mass of purified water. Stirring and mixing were performed.

A nonionic surfactant (coconut oil fatty acid diethanolamide: AMISOL CDE) of 4 pts·mass, an anionic surfactant (polyoxyethylene lauryl ether sodium sulfate: SANDET EN) of 10 pts·mass, the 10 wt %-citrate aqueous solution of 1.5 pts·mass, a glycerin of 12 pts·mass, a 1, 3-butylene glycol of 10 pts·mass, and purified water of 39.5 pts·mass together were added to Red No. 106-containing aqueous solution of 3 pts·mass at the same time. Stirring and mixing were performed.

Then, an amphoteric surfactant (amino acid system surfactant: NJBON ASP-12 TKL) of 10 pts·mass was added thereto. Stirring and mixing were performed.

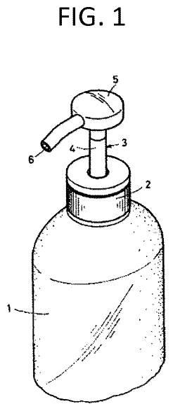

The solution was subjected to filtration by using a 100-mesh sieve. This filtrate had a pH of a range between 5.5 and 6.0. This filtrate was filled in the container body of the pump device as shown in FIG. 1.

In FIG. 1, 1 denotes a container body, 2 denotes a cap screwed in an opening of the container body 1. The cap 2 is provided with a pump device 3. When a head 5 of a piston 4 of the pump device 3 is pushed, the piston 4 moves downwardly, and thereby a lather of the solution is ejected through an outlet of a nozzle 6. Upon detaching one's hand from the head 5, the piston 4 comes up to return to the original position. This structure is a publicly known technique and, thus, a detailed description thereof is omitted here.

### Comparative Example 1A

Everything was performed in the same manner as Example 1.

Here, all the components such as the Red No. 106, the surfactant, the citrate, the glycerin, and the glycol were put into purified water at the same time (at once).

The solution was filled in the container body of the pump device of FIG. 1.

### Comparative Example 1B

Everything was performed in the same manner as Example 1.

Here, all the components other than a dye, i.e., all the components such as the surfactant, the citrate, the glycerin, and the glycol, were put into purified water at the same time (at once). Thereafter, the Red No. 106 was added to the solution.

The solution was filled in the container body of the pump device of FIG. 1.

### Examples 2-6

Everything was performed in the same manner as Example 1.

Here, instead of Red No. 106, Red No. 102 (Example 2), Yellow No. 4 (Example 3),

Yellow No. 203 (Example 4), Blue No. 1 (Example 5), and Purple No. 401 (Example 6) were employed.

Those solutions each was filled in the container body of the pump device of FIG. 1.

### Comparative Examples 2A-6A

Everything was performed in the same manner as Comparative Example 1A.

Here, instead of Red No. 106, Red No. 102 (Comparative Example 2A), Yellow No. 4 (Comparative Example 3A), Yellow No. 203 (Comparative Example 4A), Blue No. 1 (Comparative Example 5A), and Purple No. 401 (Comparative Example 6A) were employed.

Those solutions each was filled in the container body of the pump device of FIG. 1.

### Comparative Example 2B-6B

Everything was performed in the same manner as Comparative Example 1B.

Here, instead of Red No. 106, Red No. 102 (Comparative Example 2B), Yellow No. 4 (Comparative Example 3B), Yellow No. 203 (Comparative Example 4B), Blue No. 1 (Comparative Example 5B), and Purple No. 401 (Comparative Example 6B) were employed.

Those solutions each was filled in the container body of the pump device of FIG. 1.

### Example 7

Everything was performed in the same manner as Example 1.

Here, instead of AMISOL CDE, LEBON 2000 was employed. Instead of SANDET EN, LIPOFLOW MN was employed.

The solution was filled in the container body of the pump device of FIG. 1.

### Comparative Example 7A

The components of Example 7 were used, and everything was performed in the same manner as Comparative Example 1A.

The solution was filled in the container body of the pump device of FIG. 1.

### Comparative Example 7B

The components of Example 7 were used, and everything was performed in the same manner as Comparative Example 1B.

The solution was filled in the container body of the pump device of FIG. 1.

### Example 8

Everything was performed in the same manner as Example 1.

Here, instead of NJBON ASP-12 TKL, ENAGICOL DP-30 was employed.

The solution was filled in the container body of the pump device of FIG. 1.

### Comparative Example 8A

The components of Example 8 were used, and everything was performed in the same manner as Comparative Example 1A.

The solution was filled in the container body of the pump device of FIG. 1.

### Comparative Example 8B

The components of Example 8 were used, and everything was performed in the same manner as Comparative Example 1B.

The solution was filled in the container body of the pump device of FIG. 1.

### [Characteristics]

The pump device filled with the solution of the respective Example was used, and a lather of the solution was ejected through the outlet of the nozzle 6.

A result that a color of the lather which was visually observed is shown in the following Table.

| | Shading of Color |
|---|---|
| | of Lather |
| Example 1 | 5 |
| Comparative Example 1A | 2 |
| Comparative Example 1B | 2 |
| Example 2 | 4 |
| Comparative Example 2A | 2 |
| Comparative Example 2B | 1 |
| Example 3 | 4 |
| Comparative Example 3A | 2 |
| Comparative Example 3B | 2 |
| Example 4 | 4 |
| Comparative Example 4A | 2 |
| Comparative Example 4B | 2 |
| Example 5 | 6 |
| Comparative Example 5A | 2 |
| Comparative Example 5B | 2 |
| Example 6 | 5 |
| Comparative Example 6A | 2 |
| Comparative Example 6B | 2 |
| Example 7 | 4 |
| Comparative Example 7A | 2 |
| Comparative Example 7B | 1 |
| Example 8 | 3 |
| Comparative Example 8A | 2 |
| Comparative Example 8B | 1 |

A shading of color of lather in the above Table is shown by gradation (composed of 10 stages). A larger value shows a darker color.

Colors of lathers of the solutions (value: 3 or greater) of the Examples could be clearly recognized with the naked eye.

The solutions of Comparative Examples have lower values. Colors of lathers of the solutions of Comparative Examples hardly recognized with the naked eye.

### REFERENCE CHARACTER LIST

1 container body
2 cap
3 pump device
4 piston
5 head
6 nozzle (outlet)

1. A method for producing a solution for making a lather, the method comprising:
mixing a water-soluble coloring material and water to form a water-soluble coloring material-containing aqueous solution; and
adding a frothing agent to the water-soluble coloring material-containing aqueous solution.

2. The method according to claim 1, further comprising:
adding a pH adjuster to the coloring material-containing aqueous solution.

3. The method according to claim 1, further comprising:
adding a glycerin to the coloring material-containing aqueous solution.

4. The method according to claim 1, wherein the solution is neutral or weakly acidic.

5. The method according to claim 1:
wherein the frothing agent is 5-50 pts·mass to the 100 pts·mass of water; and
wherein the coloring material is 0.0001-5 pts·mass to the 100 pts·mass of water.

6. The method according to claim 1, wherein:
when shading of color is shown by gradation of 10 stages, the shading of color of the lather made by the solution is at a level deeper than the 7^{th} stage from the deeper color of the gradation;
the solution contains at least a frothing agent, a water-soluble coloring material, and water;
there exist chemically unreacted frothing agent and coloring material between the frothing agent and the coloring material in the solution; and
the solution is neutral or weakly acidic.

7. The method according to claim 1, wherein, regarding the coloring material contained in the solution, a reaction with the water lowers a reaction with the frothing agent to thereby allow existence of unreacted coloring material in the solution.

8. The method according to claim 1, wherein the frothing agent is a surfactant.

9. The method according to claim 8, wherein the surfactant is at least one selected from the group consisting of an anionic surfactant, a cationic surfactant, an amphoteric surfactant, and a nonionic surfactant.

10. (canceled)

11. The method according to claim 1, wherein the solution has a pH of a range between 7.3 and 5.5.

12. (canceled)

13. (canceled)

14. The method according to claim 1, wherein the solution is a non-aerosol type solution which uses no compressed gas upon making a lather.

15. The method according to claim 1, further comprising:
putting the solution in a container body of a device comprising the container body, an outlet, and a pump; and
operating the pump, causing the solution and the air in the container body to mix each other to eject a colored lather through the outlet.

16. The method according to claim 1, wherein, when shading of color is shown by gradation of 10 stages, the shading of color of the lather made by the solution is at a level deeper than the 7^{th} stage from the deeper color of the gradation.
